(19) United States
(12) Patent Application Publication
(10) Pub. No.: US 2008/0039870 A1
(43) Pub. Date: Feb. 14, 2008

(54) Tick Remover

(75) Inventors: Karel Golta, Hamburg (DE); Holger Prussner, Rosengarten-Klecken (DE); Gunter Uhlig, Eversen-Heide (DE)

Correspondence Address: FITZPATRICK CELLA HARPER & SCINTO, 30 ROCKEFELLER PLAZA, NEW YORK, NY, 10112, US

(73) Assignee: SOLUTIONS BRANDING & DESIGN COMPANIE GMBH & CO. KG, Hamburg (DE)

(21) Appl. No.: 10/579,091

(22) PCT Filed: Nov. 9, 2004
(86) PCT No.: PCT/EP04/12779

§ 371 Date: Feb. 6, 2007

(30) Foreign Application Priority Data
Nov. 14, 2003 (DE) 203 17 698.7

(51) Int. Cl. A61B 19/00

(52) U.S. Cl. 606/131

(57) ABSTRACT

The invention relates to an instrument (1) for pulling ticks out of the skin of animals or humans, comprising: a longitudinal grip piece (2, 21) for gripping the instrument (1) and an end piece (4), which is at least partially flattened, has, at least in part, a first slot (22), and which is provided for griping the tick. Said end piece (4) terminates in an essentially fork-like shape having at least two prongs (6) between which a space (10) for grasping a tick is formed. The invention is characterized in that the grip piece (2, 21) has a second slot (24) that permits the space (10) between the prongs (6) of the end piece to be varied.

The invention relates to an instrument for pulling ticks out of the skin of animals or humans, comprising a longitudinal grip piece for gripping the instrument and an end piece, which is at least partially flattened, having, at least in part, a first slot, and which is provided for gripping the tick, which end piece terminates in an essentially fork-like shape having at least two prongs between which a space for grasping a tick is formed.

Instruments of this kind are well known in the prior art. Thus, for example, EP 0,821,571 B1 already shows an instrument for pulling ticks out of human or animal skin. This printed source in particular shows an instrument for pulling parasitic ticks out of the skin of animals or humans having a curved and flattened end piece terminating in a fork shape, which consists of two prongs between which a space for grasping the tick is formed, where the instrument is formed in one piece and, on the one hand, comprises a pick-up section of round cross section which terminates in a grip and, on the other hand, comprises the curved and flattened end piece, where the fork shape runs at precisely right angles to the axis of the pick-up section and the instrument pulls the tick out by rotation about an axis that runs at right angles to the plane of the skin in which the tick is embedded.

The object of the present invention is to improve the instruments known in the prior art, mentioned at the beginning.

According to the invention, this object is accomplished by an instrument according to claim 1.

The advantages of the present invention lie in particular in the fact that it is possible on the basis of the present invention to vary the space between the at least two prongs of the essentially fork-like end region of the instrument. In this way, for one thing large ticks can be taken into account, in that the aforesaid space is enlarged before the tick is grasped; for another, after the instrument is put into place, in particular after the two prongs are placed on the sides of the tick, the tick can be clamped in between the two prongs by squeezing the instrument, i.e., by pressing together the slots formed in the instrument.

Thus, because of the variability of the space between the prongs, thanks to the invention only one instrument is required for all sizes of ticks.

The invention makes use of the knowledge that, because of the formation of a slot also in the grip section of the instrument, the slot provided in the end section can be compressed. In this way, very simple manipulation of the instrument, in particular compression of the end section, is possible, while at the same time positioning of the operator's hand in the grip section of the instrument allows an unobstructed view of the end section of the instrument. Therefore, when the instrument is used, the operator can tell when the end section must be compressed and when the end section has been compressed far enough that the tick can be pulled out.

Advantageously, at least one of the slots is designed V-shaped in cross section. In this embodiment, it is further preferred when the two slots are connected together by a film hinge. In this way, increased stability of the instrument is obtained.

In an additional preferred embodiment of the invention, the slot is designed continuous. This variation of the instrument is especially easy to produce. In another exemplary embodiment of the present invention, the slot, viewed in a direction parallel to the slot in the end section, has in a transition region between end section and grip section an enlarged width with respect to adjacent sections of the slot or slots. In this way, an enlargement of the width of variation of the slot in the end section is obtained.

The grip section preferably is designed as a rotary member. This permits a special advantage of the invention, namely, grasping the tick with simultaneous rotation of the instrument for releasing the tick. Owing to its symmetry with respect to rotation, when the instrument is rotated, rolling of the grip piece on the hand is possible without the hand having to reach around. This advantage of the instrument may optionally be supported by a rubber ring drawn around the grip piece.

Another preferred embodiment has a light integrated into the grip section, by which the end section, in particular the slot in the end section, can be illuminated.

In an additional preferred embodiment of the invention, a magnifying lens is mounted on the instrument in such a way that, in particular at a glance parallel to the grip section at the end section, an enlargement of the end section is obtained. In this variant, the end section can be observed better and thus the tick can be removed more easily.

Additional preferred embodiments of the invention are indicated in the sub-claims.

In the following, exemplary embodiments are now explained with reference to the accompanying drawings.

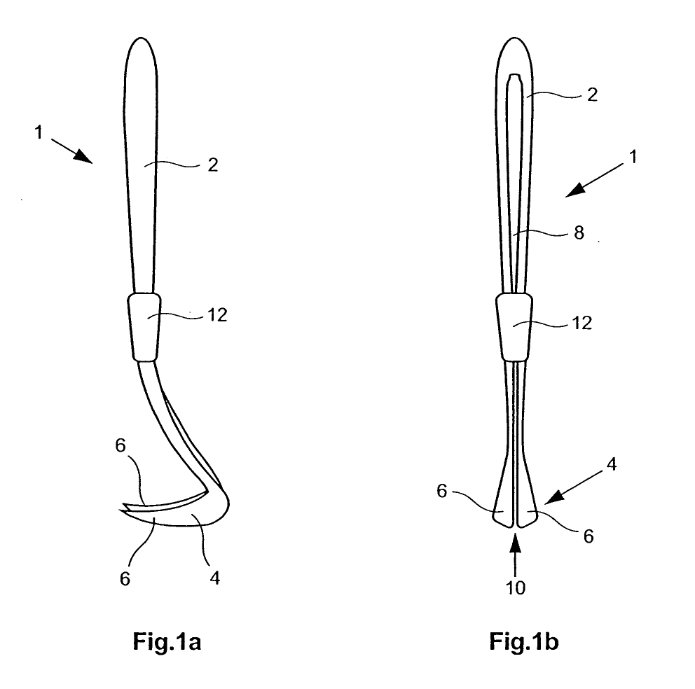

FIG. 1a shows a schematic side view of a first embodiment of the instrument according to the invention;

FIG. 1b shows a rear view of the embodiment of FIG. 1a;

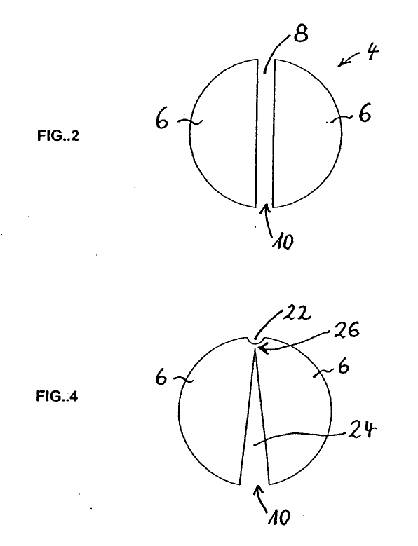

FIG. 2 shows a schematic representation of the slot in the embodiment of FIG. 1a;

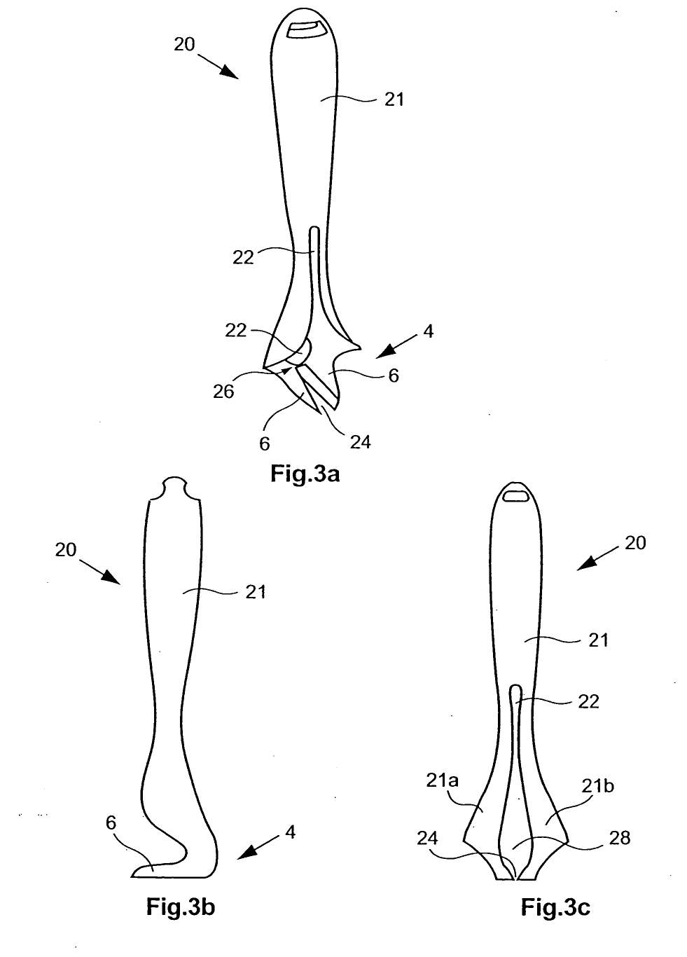

FIG. 3a shows a perspective view of a second embodiment of the instrument according to the invention;

FIG. 3b shows a side view of the embodiment of FIG. 3a;

FIG. 3c shows a front view of the embodiment of FIG. 3a; and

FIG. 4 shows a schematic representation of the slot of the embodiment of FIG. 3a.

FIG. 1a shows a side view of a first embodiment 1 of an instrument according to the invention for pulling ticks out of the skin of animals or humans. The instrument 1 has a longitudinal grip piece 2 for gripping the instrument 1 and a flattened end piece 4 for grasping the tick. The end piece 4 terminates in an essentially fork-like shape. The end piece 4 has two prongs 6, between which is formed a space 10 for grasping the tick.

The plane of grip of the at least partially flattened end piece 4 forms a right angle with the axis of rotation of the grip piece 2. The grip piece 2 is symmetrical with respect to rotation, so that rotation of the grip piece 2 permits rotation in the plane of the end piece 4 without the end piece 4 being lifted or lowered because of asymmetry in the grip piece 2. In this way, the tick can be rotated very exactly and thus removed.

Fig. 1b shows a rear view of the instrument 1 of FIG. 1a. FIG. 1b shows in particular a slot 8 formed continuous in the grip piece 2 as well as in the end piece 4. Gripping the grip piece 2 and compressing the grip piece 2 allows the space 10 formed between the prongs 6 in the end piece 4 by the slot 8 to be narrowed and the tick grasped in this way. At the same time, rotation of the instrument 1 is then possible without any change in position of the hand. Spreading open of the instrument 1 is prevented by a ring 12 made for example of rubber. However, this ring 12 serves primarily to facilitate rotation of the instrument 1 in the hand of the user upon simultaneous compression of the slot 8 by the user in order to hold the tick securely during rotation.

FIG. 2 shows a schematic representation of the space 10 between the two prongs 6 in the instrument 1 of FIGS. 1a and 1b.

FIG. 3a shows a perspective view of a second embodiment 20 of an instrument according to the invention. Parts that correspond to those of FIGS. 1a, 1b and 2 are labeled with the same reference numerals. Unlike in the first embodiment 1 of FIGS. 1a, 1b and 2, the instrument 20 of FIG. 3a has a first slot 22 formed in the grip piece 21 and a second slot 24 formed in the end piece 4. The slots 22 and 24 are connected together by a film hinge 26. The slot 24 opens V-shaped between the prongs 6 of the end piece 4. A cross section in the plane of grip of the end piece 4 through the end piece 4 is shown in FIG. 4. A lamp (not represented) illuminating the slot 24 and preferably capable of being turned on and off or a magnifying lens (not represented) may optionally be mounted in the somewhat elongated and thickened grip piece 21. The surface of the grip piece 21 is made of a non-slip material.

Again in the embodiment 20 of FIG. 3a, by compression of the grip piece 21, the slot 22 and hence, via the film hinge 26, the slot 24 can also be compressed, and so the space 10 between the prongs 6 narrowed and, lastly, the tick grasped in this way, after the two prongs 6, slightly tapered downward toward the interspace 10, have been pushed under the tick on both sides of the tick sticking in the skin. Without having to take the hand from the grip piece 21, the instrument 20 can then be rotated about the axis of rotation of the grip piece 21 in order to be able to pull the tick out of the skin more easily.

Since the slot 22 does not extend through the entire grip piece 21, unlike in embodiment 1, in embodiment 20 a ring 12 is not necessary for preventing the instrument 20 from spreading open too far.

FIG. 3b shows a side view of the instrument 20 of FIG. 3a. FIG. 3c shows a front view of the instrument 20 of FIG. 3a. It can be seen in FIG. 3c that the slot 22 in the grip piece 21, viewed in a direction essentially parallel to the first slot 24, [has] a non-uniform [width], varying essentially parallel to the longitudinal extension of the grip piece 21 and in a transition 28 between grip piece 21 and end piece 4 is enlarged with respect to sections of the slot 22 adjacent to this transition 28. In this way, compression of the two halves 21a and 21b of the grip piece 21 of the instrument 20 is facilitated.

1. Instrument (1) for pulling ticks out of the skin of animals or humans, comprising:
a longitudinal grip piece (2, 21) for gripping the instrument (1) and
an end piece (4), which is at least partially flattened, having, at least in part, a first slot (22) for gripping the tick, which end piece (4) terminates in an essentially fork-like shape, which shape has at least two prongs (6) between which a space (10) is formed for grasping a tick, characterized in that the grip piece (2, 21) has a second slot (24) in order to permit the space (10) between the prongs (6) of the end piece (4) to be varied.

2. Instrument (1) according to claim 1, where at least one of the slots (8, 22, 24) has an essentially V-shaped cross section (10).

3. Instrument (1) according to either of the preceding claims, where the slots (8, 22, 24) are connected together by a film hinge.

4. Instrument (1) according to any of the preceding claims, where the first slot (22) and the second slot (24) are designed as one slot (8).

5. Instrument (1) according to any of the preceding claims, where the second slot (24), viewed in a direction essentially parallel to the first slot (22), has a non-uniform width.

6. Instrument (1) according to the preceding claim, where the slot width varies essentially parallel to the longitudinal extension of the grip piece (2, 21).

7. Instrument (1) according to the preceding claim, where the slot width, at least in a transition (28) between grip piece (2, 21) and end piece (4), is enlarged with respect to sections of the slot (8, 22, 24) adjacent to this transition (28).

8. Instrument (1) according to any of the preceding claims, where the grip piece (2, 21) is symmetrical with respect to rotation.

9. Instrument (1) according to any of the preceding claims, where a light source is provided, by which the end piece (4) is capable of being at least substantially illuminated.

10. Instrument (1) according to any of the preceding claims, where a magnifying lens directed to the end piece (4) is provided.
